(19) United States
(12) Patent Application Publication
(10) Pub. No.: US 2011/0144207 A1
(43) Pub. Date: Jun. 16, 2011

(54) ARYL ALKYL CARBOXYLIC ACID SALTS, PROCESS FOR PREPARATION AND DOSAGE FORMS

(75) Inventors: Nandkumar Chodankar, Goa (IN); Milind Biyani, Mumbai (IN); Mohan Muthunarayanan, Pondicherry (IN); Selvaraju Radhakrishnan, Tamil Nadu (IN); Sathish Kumar Santhanampillai, Tamil Nadu (IN); Rajendran Paul Nadar, Puducherry (IN); Vivekanandan Sundaramurthy, Tamil Nadu (IN); Sakthivel Lakshamana Prabu, Tamil Nadu (IN)

(73) Assignee: SHASUN CHEMICALS AND DRUGS LIMITED, Chennai (IN)

(21) Appl. No.: 13/058,978

(22) PCT Filed: Aug. 14, 2009
(86) PCT No.: PCT/IB2009/053592

§ 371 Date: Feb. 14, 2011

(30) Foreign Application Priority Data
Aug. 14, 2008 (IN) 1977/CHE/2008

(51) Int. Cl. A61K 31/19; C07C 63/00; C07C 63/04; C07C 65/24; C07C 229/40; C07D 209/26; C07D 491/052; C07C 63/33; C07C 59/185; A61P 29/00

(52) U.S. Cl. 514/568; 562/405; 562/493; 562/466; 562/456; 548/501; 548/432; 562/492; 562/460

(57) ABSTRACT

The invention particularly discloses a process for preparing aryl alkyl carboxylic acid salts by preparing aqueous alkali solution, adding aryl alkyl carboxylic acid to said alkali solution at a temperature ranging from 4° to 121° C. for obtaining a clear solution, preferably by heating and/or stirring and concentrating and cooling to obtain aryl alkyl carboxylic acid salt The invention therefore discloses solid oral dosage forms and compositions of aryl alkyl carboxylic acid salts which are free of organic solvent/so. The solid oral dose compositions of aryl alkyl carboxylic acid salts of the invention are prepared in situ from aryl alkyl carboxylic acids and bases to obtain aryl acid alkyl carboxylic acid sails in crystalline/powder form with or without the use of pharmaceutical excipients.

### FIELD OF THE INVENTION

The present invention relates to the preparation of aryl alkyl carboxylic acid salts and formulations, both from aryl alkyl carboxylic acids. In particular, this invention relates to oral solid dosage forms of aryl alkyl carboxylic acid salts prepared directly from aryl alkyl carboxylic acids.

### DESCRIPTION OF PRIOR ART

Aryl alkyl carboxylic acids are illustrated by examples such as ibuprofen, (±)-2-(4-isobutylphenyl)propionic acid; naproxen, d-2-(6-methoxy-2-napthyl)propionic acid; diclofenac 2-[(2,6-Dichlorophenyl)-amino]benzeneacetic acid; indomethacin, 1-(4-Chlorobenzoyl)-5-methoxy-2-methyl-1H-indole-3-acetic acid; etodolac, 1,8-Diethyl-1,3,4,9-tetrahydropyrano [3,4-b]indole-1-acetic acid; flurbiprofen, 2-(2-fluoro-4-biphenylyl)propionic acid; and ketoprofen, 2-(3-benzoyl-phenyl)propionic acid; and its active enantiomers such as dexibuprofen in the case of ibuprofen.

Salts of these acids have higher solubility and faster onset of action when compared to their acid forms. For example, ibuprofen sodium dihydrate has a significantly higher Cₘₐₓ (peak concentration) and earlier tₘₐₓ (time to peak concentration) than the conventional ibuprofen.

U.S. Pat. No. 4,859,704 describes water-soluble ibuprofen compositions and methods of making them. An alkali metal salt of ibuprofen was prepared by treating ibuprofen with alkali metal carbonate in aqueous media at 55-60° C. Crystalline alkali metal salts of ibuprofen were obtained by evaporation at high temperature or by exposing the solution to vacuum or by freeze drying process. Alkali metal salts used are bicarbonate of sodium and potassium. The invention discloses the preparation of ibuprofen alkali salts and its formulation as tablet and aqueous solution. For the preparation of tablets, sodium or potassium salts are first obtained in the powder form. The main drawback of the invention process is foam formation due to carbon dioxide evolution.

U.S. Pat. No. 5,019,563 discloses cyclodextrin complexes with ibuprofen salts, their preparation and pharmaceutical compositions. The prepared complexes were used in the form of powder, granules or tablets.

U.S. Pat. No. 5,043,167 describes galenic formulations with programmed release. The invention discloses the procedure for preparation of a galenic programmed release formulation using naproxen sodium or diclofenac sodium for oral use. The first process involves mixing of sodium naproxen or diclofenac sodium, sodium starch glycolate, maize starch, polyvinylpyrrolidone, lactose and magnesium stearate and wet granulated with ethyl alcohol. The second process involves mixing of sodium naproxen or diclofenac sodium, hydrogenated castor oil and ethylcellulose and wet granulated with ethyl alcohol. The two granules are carefully mixed and the resulting mixture is compressed into tablets.

EP application 91300182.2 describes the formation and resolution of ibuprofen lysinate. The process employs preferential crystallization to separate the diasteromeric salts. The process involves addition of ibuprofen, lysine with water and ethanol, and the resulting slurry agitated for 24 hours. The resulting clear liquor was filtered; the supersaturated liquor was then added to a slurry of (S)-ibuprofen-(S)-lysine. The precipitated solid was separated by filtration and the mother liquor was recycled. This process is a time consuming one.

EP 478838 claims a preparation having improved tabletting capacity, which contains ibuprofen and/or S-(+)-ibuprofen as well as drug additives and/or drug vehicles, characterized in that it contains 50 to 100% by weight of calcium salt of ibuprofen and/or the calcium salt of S-(+)-ibuprofen.

U.S. Pat. No. 5,260,482 describes a process for the preparation of an enantiomerically enriched hydrated salt of the carboxylic acid. The process involves addition of sodium hydroxide to ibuprofen in hexane and heated to 65° C., then cooled to 5° C. The solution was seeded with sodium salt and agitated for 16 hours at 5° C., followed by the addition of water to induce precipitation. The resulting salt was isolated by filtration.

U.S. Pat. No. 5,262,179 describes non-effervescent water soluble ibuprofen compositions. The process involves the mixing of ibuprofen salt, sodium bicarbonate, dextrose, sodium saccharin and flavor.

U.S. Pat. No. 5,470,580 describes directly compressible naproxen or naproxen sodium compositions. The process involves mixing of naproxen or naproxen sodium with croscarmellose sodium, povidone and water. The mixture is spray-dried, mixed with magnesium stearate and compressed into tablets. The invention claims a directly compressible naproxen composition consisting of 90 to 97% spray dried naproxen, free moisture, binder, disintegrating agent and lubricant and also describes a process for preparing a tablet thereof.

U.S. Pat. No. 5,512,300 describes prevention of ibuprofen from forming low melting eutectics with other therapeutic agents in solid dosage forms. The process involves directly heating ibuprofen to form a melt; combining said ibuprofen melt with an alkali metal to forge an amalgamation, following which the mixture is blended and cooled to room temperature. Granulation was made by passing through # 30 mesh screen. The invention claims a method of stabilizing ibuprofen.

U.S. Pat. No. 5,696,165 discloses a solid or semi-solid pharmaceutical composition comprising at least 90% of S(−)sodium 2(4-isobutylphenyl)-propionate together with a pharmaceutically acceptable carrier.

U.S. Pat. No. 5,702,724 describes the process for the preparation of an oral solid dosage form containing diclofenac. The invention discloses the procedure for preparation of compressed tablet containing diclofenac or its salt, which comprises preparing an inclusion compound consisting of diclofenac salt with γ-cyclodextrin, and also describes a process for the preparation of dragees and the preparation of an inclusion compound comprising γ-cyclodextrin. The process involves mixing of diclofenac salt and γ-cyclodextrin in water and allowing to cool to room temperature, while crystals are precipitated. Crystals were further isolated by centrifuation and washed with ice-water and dried at 40° C. This inclusion compound was used for the formulation of tablets with acceptable excipients.

U.S. Pat. No. 5,711,967 describes oral diclofenac preparation. The process involves coating of inert pellets initially with an active ingredient layer, secondly by a membrane layer followed by film coating to produce the controlled release formulation. The invention disclose a pelleted oral drug preparation comprising an active ingredient layer such as diclofenac or one of its salts, with controlled release of the active ingredient.

U.S. Pat. No. 5,969,181 describes the preparation of salts of pharmaceutical active substances which have acidic groups. The invention discloses a process for preparing salts of pharmaceutically active substances having acidic groups, obtained by reacting the carboxylic acids with a base in the melt. The process involves addition of sodium carbonate with ibuprofen in an extruder in the presence of an organic solvent as an entrainer (cyclohexane, toluene, petroleum ether and lower molecular weight alcohols). The extruder was operated up to 40 hrs when ibuprofen was completely converted into the sodium salt. Product obtained from extrudate consisted of coarse particle granules. This process is a time consuming one.

U.S. Pat. No. 6,224,911 describes process for the preparation of enteric coated pharmaceutical dosage forms. The process involves preparation of aqueous enteric coating dispersion and coating on the previously seal coated naproxen sodium tablets.

U.S. Pat. No. 6,242,000 describes composition of S(−)sodium ibuprofen. The invention claims S(−)sodium 2(4-isobutylphenyl)propionate dihydrate having an enantiomeric purity of at least 90%.

U.S. Pat. No. 6,525,214 discloses the process for producing a substantially pure enantiomeric salt by reacting 2-(4-isobutylphenyl)propionic acid enriched with one of its enantiomers with a sodium containing base thereby forming a sodium salt of 2-(4-isobutylphenyl)propionic acid, treating said salt with an organic solvent and separating the sodium salt of the pure salt. This patent also describes the procedure for the preparation of the hydrated salt of S(−)sodium ibuprofen.

U.S. Pat. Appl. 2002/0034540 describes dosage forms of ibuprofen. The invention disclose preparation procedure of a solid non-effervescent compressed solid dosage form comprising ibuprofen medicament. The process involves mixing of ibuprofen sodium salt dihydrate, microcrystalline cellulose (PH102)/(PH101) with lactose, anhydrous sodium carbonate, croscarmellose sodium, colloidal silicon dioxide, stearic acid and magnesium stearate and then compressed into tablet.

EP application 1149828 describes crystalline form of diclofenac sodium salt. The invention discloses the procedure for obtaining diclofenac sodium salt hemihydrate which has no hygroscopic property, whereas diclofenac sodium salt is hygroscopic.

U.S. Pat. Appl. 2003/0211150 describes immediate release tablet containing naproxen sodium. The invention discloses the procedure for preparation of immediate release of naproxen sodium comprising naproxen sodium and spray-dried mannitol. The process involves mixing of naproxen sodium, spray dried mannitol and sodium stearyl fumarate, then compressed into tablet.

U.S. Pat. Appl. 2004/0102522 describes dosage forms of sodium ibuprofen. This invention discloses non-effervescent tablet for oral administration comprising a tablet core (50-100% by weight of ibuprofen sodium hydrate) with a sugar or film coat and water content from 8-16% by weight. However the inventors of the present invention found it difficult to produce sufficiently hard tablets particularly when the water content of the sodium ibuprofen hydrate is less than 11%. It was further more observed that the hardness and disintegration time of the tablets as mentioned in this application were nearly independent of the compressive force used during tabletization.

U.S. Pat. No. 7,084,299 describes the process for producing ibuprofen sodium dihydrate. The process involves dissolving ibuprofen in tetrahydrofuran, stirring with long chain carboxylic acid like sodium 2-ethyl hexanoate for 1 hr at 28° C. and then for 6 hrs to evaporate the solvent. Final product is obtained by precipitating the resulting product in desired quantity of acetone. The invention claims a process for preparing sodium dihydrate salt of ibuprofen.

WO 2006/100281 (HP 1863460 A2) describes solubilized ibuprofen. The invention discloses the process for producing a solubilized ibuprofen, preferably in the form of a granulate. The process involved in the invention is the physical mixing of ibuprofen with various salts and does not involve the preparation of ibuprofen salt. The process involves mixing of ibuprofen, sodium hydroxide, potassium hydroxide, sodium carbonate, potassium carbonate, sodium glycinate, potassium glycinate and tribasic sodium and potassium phosphates and mixtures, resulting in solubilized ibuprofen as granules, wherein these granules can be processed further to tablets.

U.S. Pat. Appl. 20070184100 and PCT application WO 2007/092784 describe compositions containing solid ibuprofen concentrates and methods of making solid ibuprofen concentrates. Said solid ibuprofen concentrates comprises solid ibuprofen free acid and solid ibuprofen alkali salt wherein at least 90% of the weight of the solid ibuprofen concentrate is ibuprofen free acid and ibuprofen alkali salt. The process involves dry mixing of ibuprofen with potassium bicarbonate and potassium carbonate and mixed with aqueous potassium hydroxide solution and dried at 45±5° C. Dried ibuprofen concentrate was then passed through 40 mesh screen. These granules were used for the various formulations hard shell capsule, soft gelatin capsule, liquid suspension, liquid solution and tablet.

U.S. Pat. Appl. 20070254028 describes the granules comprising a non-steroidal anti-inflammatory drugs (NSAID) and a sugar alcohol made by melt extrusion. The invention discloses a pharmaceutical composition comprising a granular component comprising a plurality of solidified melt granules of sugar alcohol having a salt of a NSAID salt and also describes a composition comprising a water insoluble wicking. The process involves dry blending the sugar alcohol with NSAID salt, and heating it to 100-165° C., which is further cooled and passed through a cone mill having a screen with a round hole of 1 mm, where granules are collected. Granules were used for the formulations like effervescent formulation, chewable tablets, powder mixture and non-effervescent compressed tablet.

WO 2007/035448 describes the preparation of highly concentrated pourable aqueous solutions of potassium ibuprofen and their uses. The process involves the addition of PEG 400 flakes, ibuprofen and potassium salt of ibuprofen in a round bottom flask containing specific quantity of water and ethanol as solvent and heating the content to about 70° C., while part of the solvent was evaporated by vacuum. The final product composed of ibuprofen, potassium ibuprofen, water, ethanol and PEG.

U.S. Pat. Appl. 20070265344 describes non-steroidal anti-inflammatory oral powder and liquid preparations for administration to animals. The invention claims a dry formulation comprising a NSAID, a base of an amino acid, and a strong base. The first process involves mixing of ibuprofen powder, L-Arginine, sodium hydroxide, sodium saccharin, cherry liquid and deionized water to form an aqueous ibuprofen solution. Second process involves mixing of L-Arginine, Ibuprofen, disodium phosphate, sodium saccharin and cherry powder to form a dry uniform ibuprofen mixture.

WO 2008/024820 describes process for the preparation of sodium salt of ibuprofen of controlled median particle sizes. The process involves dissolving ibuprofen in hexanes and maintaining at 60.5° C., while aqueous sodium hydroxide was added drop wise. Free water was distilled from the reactor until all the theoretical amount of water was collected. The slurry was then cooled to about room temperature and left stagnant for 60 hrs. The solids were isolated by centrifugation and washed with hexane.

WO 2008/037557 describes solubilized non-steroidal anti-inflammatory drugs. The invention discloses the procedure for preparation of solubilized NSAID drugs as granules. The process involves mixing of NSAID, sodium hydroxide, potassium hydroxide, sodium carbonate, potassium carbonate, sodium glycinate, potassium glycinate and tribasic sodium and potassium phosphates and mixtures, resulting the solubilized NSAID as granules, these granules can be processed further to tablets.

It will be observed that the references identified above uses organic solvents to prepare sodium or potassium salts from alkali hydroxides and other bases. The main drawback of the above mentioned inventions is the use of organic solvents during the process since one has to dry the final product extensively due to the required low levels of solvent in the final dosage form. From a process point of view one would have remarkably less environmental problems if the organic solvent could be circumvented. Moreover, none of the references describe the preparation of solid dosage forms of aryl alkyl acid salts directly from aryl alkali carboxylic acids.

In view of the foregoing limitations, there remains a need for methods of preparing the non-steroidal anti inflammatory drug salts and its dosage forms, as well as oral non-steroidal anti inflammatory drug products with high drug concentration. In conducting research for a way to fulfill this need, a process was found which not only achieves that objective, but which additionally enables the production of salts of non-steroidal anti inflammatory drug and its dosage form.

This application is the enabling disclosure of alkali metal salt of non-steroidal anti inflammatory drug and its dosage form a process of making them. The invention disclosed herein demonstrates simple, economical and a commercially valuable process for producing alkali metal salt of non-steroidal anti inflammatory drug and its dosage form using suitable salt and water.

### SUMMARY OF THE INVENTION

It is a principal object of the invention to prepare aryl alkyl carboxylic acid salts from aryl alkyl carboxylic acids and corresponding bases in aqueous media without the use of organic solvents so that the product is made in a cost effective manner and is environmentally friendly.

The invention particularly discloses a process for preparing aryl alkyl carboxylic acid salts by preparing aqueous alkali solution, adding aryl alkyl carboxylic acid to said alkali solution at a temperature ranging from 4° to 121° C. for obtaining a clear solution, preferably by heating and/or stirring and concentrating and cooling to obtain aryl alkyl carboxylic acid salt. The invention therefore discloses solid oral dosage forms and compositions of aryl alkyl carboxylic acid salts which are free of organic solvent/s. The solid oral dose compositions of aryl alkyl carboxylic acid salts of the invention are prepared in situ from aryl alkyl carboxylic acids and bases to obtain aryl acid alkyl carboxylic acid salts in crystalline/powder form with or without the use of pharmaceutical excipients.

It is another object of the present invention to provide the aryl alkyl carboxylic acid salts in hydrated and anhydrous forms. Hydrated form may be monohydrate or dihydrate.

It is still another object of the invention to prepare dosage forms of aryl alkyl carboxylic acid salts directly from aryl alkyl carboxylic acids in-situ in the absence of any organic solvents without isolation of the final compound.

It is another object of the present invention to prepare intermediate compositions of aryl acids carboxylic salts suitable for the preparation of dosage forms. These intermediate granules or powder can also be used as such by filling in sachets or capsules. Optionally these salts can be compacted or slugged with or without additional excipients.

Another object of the invention is to provide compositions of these salts which can he efficiently compressed in to tablets or filled in capsules and sachets.

Still another object of the invention is to provide better attributes of intermediates for formulation by combining excipients with the solution of salts.

Another object of the invention is to provide stable compositions of these salts.

Thus according to the basic aspect of the present invention, there are provided compositions of aryl alkyl carboxylic acid salts prepared directly from corresponding alkyl aryl carboxylic acids, and processes for the preparation of such salts in aqueous media without the use of organic solvents, thereby providing environmental friendly processes. Aryl alkyl carboxylic acids include, but are not limited to, Ibuprofen, Naproxen, Diclofenac, Indomethacin, Etodolac, Flurbiprofen, Ketoprofen and their optically active enantiomers.

Salts comprise bases or alkalis of sodium, potassium, calcium, zinc, lysinate, arginate, glycine and such amino acids and amines which forms salts with acids mentioned above. These may be in the form of oxides, hydroxides or carbonates or bicarbonates.

Compositions comprise aryl alkyl carboxylic acid salts, inert fillers, binders, disintegrants and lubricants.

According to another aspect of the invention there is provided a process for preparation of intermediates which can be used as directly compressible granules or powders for preparation of solid dosage forms. Excipients are selected to provide these attributes to the products.

In accordance with yet another aspect of the invention, there are provided compositions of dosage forms which are stable for all applicable physical as well as chemical parameters.

### DETAILED DESCRIPTION OF THE INVENTION

The subject invention is directed to the preparation of aryl alkyl carboxylic acid salts and their dosage forms from the corresponding carboxylic acids and bases.

Preparation of these aryl alkyl carboxylic acids salts using sodium or potassium hydroxide is known wherein the organic solvents are used for dissolving aryl alkyl carboxylic acids or for precipitation of salts. The product is recovered by crystallization process and leaves supernatant containing solvents and this to be separated to get pure API. Present invention does not make use of any organic solvent. The process is designed in such a way that there is complete salt formation without using excess of the base and the salt so formed is recovered completely.

Alkali solutions are prepared in water. The quantity of alkali taken is equimolar to Aryl Alkyl Carboxylic Acid to be used for the salt preparation. The quantity of water taken is just enough to dissolve the alkali and resulting salt at selected given temperature, wherein the temperature may more preferably vary from about 4° C. to about 121° C. depending on the base or acid used. The selected temperature may vary from 4° C. to 121° C. depending upon the base or acid. The alkali solution is filtered to remove particles, if any. The acid is added to the alkali. Optionally if the acid is low melting it can be melted and to this alkali solution is added. Heating and stirring is continued till the solution is clear. After completion of the reaction, the solution is either concentrated or spray dried or allowed to cool. This step is preferably carried out under slow stirring. Cooling temperature varies from 0° C. to 25° C. depending upon the salt.

The salt may be crystalline and can be filtered or centrifuged. The mother liquor can be recycled for next reaction in which only required amounts of alkali and acid are added. Still better option is to remove part of the water by heating, till it is concentrated to such extent that once it is cooled to about 30° C. the material is almost semi solid which can be wet milled or dried partially in a controlled manner or completely in a fluid bed or tray drier or spray dried to obtain the salts in hydrated/anhydrous form. Hydrated form may be monohydrate or dihydrate. The dried mass is milled to desired particle size and sieved. The above described conditions afford a palatable, free flowing, pharmaceutically acceptable powder, which is soluble in water and does not absorb further water. The resulting aryl alkyl carboxylic acid salts are used for preparation of directly compressible grade powders and granules and other solid dosage forms. The above approach also cuts down several steps such as crystallization, separation and drying.

The final composition of this invention contains no organic solvents, and it can be converted into pharmaceutical preparations for human use. In other words, the final pharmaceutical product for internal human consumption does not contain any organic solvents.

An aryl alkyl carboxylic acid salts in accordance with the invention has the advantages that it is water soluble, that the analgesic is readily taken up by the body so that its effects may be obtained rapidly and effectively. Also, it is believed that less gastrointestinal distress results from the use of the alkali metal salts of aryl alkyl carboxylic acid rather than aryl alkyl carboxylic acid itself. The invention permits the preparation of aryl alkyl carboxylic acid alkali metal salts that have pH close to neutral and do not interfere with the natural pH balance, especially in the stomach and the rest of the gastrointestinal tract.

The present invention includes a method of preparing aryl alkyl carboxylic acid salts as hydrated/anhydrous form. Hydrated form may be monohydrate or dihydrate. These salts can be incorporated into several solid formulations.

Solid dosage forms such as granules, powders, tablets and capsules of such aryl alkyl carboxylic acid salts are reported. These are prepared using aryl alky carboxylic acid salts and excipients. The present invention discloses better option of making these dosage forms directly from aryl alkyl carboxylic acids. Appropriate excipients are added to the solution or suspension/suspension/slurry or paste of the salt as prepared above. These provide intimate mixing of the active drug with excipients thereby imparting better pharmaceutical attributes. These include better flow, higher compressibility, non-sticking and better stability. This gives better in-vitro dissolution and therefore better bioavailability.

Optionally these salts can be compacted or slugged with or without excipients and compressed into tablets with or without additional excipients.

The major problems of developing pharmaceutical formulations of NSAID's are to address its poor solubility in water. Salts of these drugs are freely soluble in water than the corresponding free acid. Therefore, it is a good candidate for preparing a pharmaceutical formulation having better bioavailability. They permit preparation of pharmaceutical composition which is virtually lacking in the unpleasant taste sensations of NSAID, and they are stable in aqueous media. There has been a long felt need for an analgesic composition which is safe, effective and capable of being formed into a pharmaceutically elegant product. Solid compositions for oral administration are preferred compositions of the invention and there are known pharmaceutical forms for such administration, for example tablets and capsules.

The above approach also cut downs several steps such as crystallization, separation and drying. It also excludes granulation step wherever applicable.

For powder dosage forms, excipients are selected from the group of diluents, binders, disintegrants, lubricants and glidants. Diluents selected are from Starch, Microcrystalline cellulose, Lactose, Calcium phosphates or carbonates or sulphates, sugars and their derivatives. Binders are starch and their derivatives, povidones, gums, cellulose and their derivatives and sugars. Disintegrants include cellulose, starch derivatives and resins. Lubricants used are talc, stearic acid and its salts, glyceryl behenate, hydrogenated vegetable oils and colloidal silica dioxide. Glidants used are silica derivatives, talc and corn starch. The tablets are coated using commonly used coating materials. Since the production of the tablets can be carried out with the conventional tablet process, the proportion of auxiliary materials can be kept low, and the active ingredient costs are low, the production of tablets of this invention is particularly economically feasible. This invention enables a significant decrease of the tablet weight and size. It also exclude granulation step wherever applicable.

Directly compressible granules or powder as obtained above are filled in sachets or capsules or compressed into tablets as such or after blending with additional excipients.

The details of the invention, its object and advantages are explained hereunder in greater details by way of examples and it is to be understood that the invention, as fully described herein is not intended to be limited by the examples mentioned herein.

### EXAMPLES

### Example 1

### Ibuprofen Sodium Dihydrate Salt Preparation

2.0 kg of sodium hydroxide was dissolved in 10.0 kg of water, heated up to 70° C.-75° C. To this 10.0 kg of ibuprofen was added with continuous stirring. Heating was continued at 70° C.-75° C. and maintained till complete neutralization and dissolution. After getting clear solution the excess water was evaporated, so that the moisture content in the mass is not more than 30%. The reaction mass is dried in fluid bed drier without exceeding outlet temperature 32° C., till moisture content lie between 14.0% and 15.0%. The yield was found to be 12.0 kg (93.8%).

### Example 2

### Dexibuprofen Sodium Dihydrate Salt Preparation

10 g of sodium hydroxide was dissolved in 50 g of water at the temperature around (25° C.-60° C.). To this 51.58 g of dexibuprofen was added with continuous stirring. After getting clear solution the water was evaporated, so that the moisture content in the mass is not more than 30%. The reaction mass is dried in fluid bed drier without exceeding outlet temperature 32° C. till moisture content lie between 14.0% and 15.0%. The yield was found to be 57 g (86.4%).

### Example 3

### Ibuprofen Sodium Dihydrate Granule Preparation

40 g of sodium hydroxide was dissolved in 180 g of water, heated up to 70° C.-75° C. To this 206 g of ibuprofen was added with continuous stirring. After getting clear solution 26 g of starch was added and mixed thoroughly. Then the water was evaporated, so that the moisture content in the mass is not more than 30%. The reaction mass is dried in fluid bed drier without exceeding outlet temperature 32° C. till the moisture content lie between 14.0% and 15.0%. The yield was found to be 266 g (91.7%). The granules can be used for the preparation of oral solid dosage form such as tablets and capsules.

### Example 4

### Tablet Preparation

These granules obtained by example 3 were blended with 10 g microcrystalline cellulose 102, 5 g sodium starch glycolate, 3 g cabosil and 5 g talc. The granules were compressed into tablets of 700 mg average weight (each tablet containing ibuprofen sodium dihydrate equivalent to 400 mg of ibuprofen).

### Example 5

### Ibuprofen Sodium Dihydrate API Preparation

25 g of racemic Ibuprofen was taken in Round Bottom Flask (RBF) and heated up to 50-55° C. 5 g of sodium hydroxide was dissolved in 50 g of water and filtered. Then sodium hydroxide solution was added to 50-55° C. heated racemic ibuprofen in RBF and then heated till temperature reached 70-75° C. The temperature of 70-75° C. was maintained for 30 mins and ensured the clarity of solution by adjusting the pH between 8.0 and 9.0. A Buchi mini spray drier (B-290) was used for spray drying of reaction mass. Pump 1 was connected to purified water and pump 2 was connected to the reaction mixture and both pumps set at the rate of 1 to 2%, Then the reaction mass at 70-75° C. was sprayed at inlet temperature of 70-75° C., outlet temperature of 55-60° C. The spray drying was continued till spray completed quantity of reaction mass. The reaction mass was dried without exceeding outlet temperature 55-60° C. till the moisture content lie between 12.5% and 14.50%. The API achieved mean particle size was around 210 to 310 micron.

### Example 6

### Manufacture of Tablets Using API Obtained From Example 5 (Batch Size: 0.69 kg)

### Binder Preparation:

Starch paste prepared by mixing slurry of starch paste (prepared by dispersing Maize starch 20 mg (5.62%) in equal quantity of purified water) in boiling purified water of 13 times that of starch quantity, under continuous stirring, to form a thin translucent paste and then the paste was allowed to cool to 50° C. Lactose slurry was prepared by dispersing 20 mg (5.62%) in equal quantity of purified water, and was added to the above 50° C. cooled paste under continuous stirring.

### Ibuprofen Sodium DC Granules Preparation:

Ibuprofen sodium dihydrate 256.2 mg (72.03%) sifted through #20 mesh ASTM was dry mixed with #40 mesh ASTM sifted Sodium starch glycollate 5 mg (1.41%) in fluid bed processor for 10 mins. The paste was sprayed to the above dry mix using top spray assembly in fluid bed processor at an inlet temperature of 50±5° C., bed temperature of 35±5° C., at a spray rate of 2 to 20 gm/minute, with the atomization of around 1.5 to 2 kg/cm². Top spray process was continued, till completion of binder. Final dried granules were sifted through #16 mesh ASTM. Final dried sifted granules of 301.2 mg(84.68%) was blended with co-sifted (#40 mesh ASTM) materials of sodium starch glycollate 20 mg (5.62%) and anhydrous colloidal silica 2 mg (0.56%) in a 5L Y-blender at 25 RPM for 3 mins. Then the above blend was lubricated using #40 mesh ASTM sifted materials of Talc 15 mg (4.22%) and Magnesium stearate 17.5 mg (4.92%) for 2 mins at 25 RPM.

### Ibuprofen Sodium Tablets Compression and Coating:

The above lubricated blend was compressed for average weight of 355.7 (equivalent to 200 mg of ibuprofen) using 10.32 mm deep concave plain punches with the thickness of around 5.50±0.1 mm with the hardness of around 40±20N to get D.T of around 10 min 39 secs.

The above lubricated blend was compressed for average weight of 711.4 (equivalent to 400 mg of ibuprofen) using 16.0×8.0 mm caplet shaped plain punches with the thickness of around 6.10±0.1 mm with the hardness of around 105±25N to get D.T of around 14 min 32 secs.

The tablets were film coated using conventional coating pan using 10% w/w concentration coating suspension, to get the weight build up of 3.0% w/w.

While the present invention has been described with respect to certain preferred embodiments, it will be apparent to those skilled in the art that various changes and modifications may be made without departing from the scope of the invention as defined in the following claims

1. A process for preparing aryl alkyl carboxylic acid salts comprising the steps of:
a. Preparing aqueous alkali solution,
b. Adding aryl alkyl carboxylic acid to said alkali solution at a temperature ranging from 4° to 121° C.,
c. Obtaining a clear solution, preferably by heating and/or stirring,
d. Concentrating the clear solution preferably by drying,
e. Cooling at 0° to 25° C. cooling temperature said concentrated solution to obtain aryl alkyl carboxylic acid salt.

2. The process as claimed in claim 1 further comprising the step of preparing granule, powder, tablet or capsule solid dosage form of aryl alkyl carboxylic acid salt with or without excipients.

3. The process as claimed in claim 2—wherein said granules are formed by milling and sieving of the dried aryl alkyl carboxylic acid salt.

4. The process as claimed in claim 1 wherein said aqueous alkali solution of step a is prepared by mixing water sufficient for dissolving the alkali and the resulting aryl alkyl carboxylic acid salt.

5. The process as claimed in claim 1 wherein said alkali of step a comprise of bases of sodium, potassium, calcium, zinc, lysinate, arginate, glycine and such amino acids and amines which forms salts with aryl alkyl carboxylic acids, preferably in the form of oxides, hydroxides or carbonates or bicarbonates.

6. The process as claimed in claim 1 wherein said aryl alkyl carboxylic acid of step b is low melting acid and said alkali solution of step a is added to the melted aryl alkyl carboxylic acid.

7. The process as claimed in claim 1 wherein said aryl alky carboxylic acid of step b include Ibuprofen, Naproxen, Diclofenac, Indomethacin, Etodolac, Flurbiprofen, Ketoprofen and their optically active enantiomers.

8. The process as claimed in claim 1 wherein said concentrating in step d is by heating to remove excess water to obtain semi-solid hydrated form of aryl alkyl carboxylic acid salt.

9. The process as claimed in claim 1 wherein said drying step of step d is by spray draying or fluid bed or tray drying.

10. The process as claimed in claim 1 wherein said salt in step e is preferably obtained by filtration or centrifugation as crystalline salt and optionally the mother liquor is recycled.

11. The process as claimed in claim 1 wherein said solid dosage form of step 2 is prepared by addition of excipients to the solution or suspension/slurry or paste of the aryl alkyl carboxylic acid salt.

12. Aryl alkyl carboxylic acid salts as and when prepared by the process of claim 1.

13. Solid oral dosage forms and compositions of aryl alkyl carboxylic acid salts wherein said forms and/or compositions are free of organic solvent/s.

14. Solid oral dose compositions of aryl alkyl carboxylic acid salts comprising
a. Aryl alkyl carboxylic acid salts prepared in situ from aryl alkyl carboxylic acids and bases
b. Obtaining aryl acid alkyl carboxylic acid salts in crystalline/powder form
c. Optionally adding pharmaceutical excipients in situ in step a.
